(19) United States
(12) Patent Application Publication
(10) Pub. No.: US 2025/0157675 A1
(43) Pub. Date: May 15, 2025

(54) METHOD AND SYSTEM FOR AUTOMATICALLY GENERATING TEMPLATE-BASED PATIENT NOTES

(71) Applicant: Cloudphysician Healthcare Pvt Ltd, Bangalore (IN)

(72) Inventors: Dileep C. Unnikrishnan, Palakkad (IN); Dileep Raman, Bengaluru (IN); Dhruv Sud, Bengaluru (IN)

(21) Appl. No.: 18/942,852

(22) Filed: Nov. 11, 2024

(30) Foreign Application Priority Data
Nov. 10, 2023 (IN) 202341077058

(51) Int. Cl. G16H 70/20; G16H 10/60

(52) U.S. Cl. CPC G16H 70/20; G16H 10/60

(57) ABSTRACT

This disclosure relates to method and system for automatically generating template-based patient notes. The method includes receiving patient data from Electronic Medical Records (EMR) of the patient and an audio input corresponding to a patient from a healthcare provider. The method further includes generating primary insights text data from the audio input through a speech-to-text conversion model to obtain a plurality of unique natural language sentences. The method further includes assigning primary category and at least one secondary category associated with the primary category to each of the unique natural language sentences through a set of Machine Learning (ML) models. The method further generates a final note corresponding to the patient based on a note template.

### TECHNICAL FIELD

This disclosure relates generally to the field of healthcare, and more particularly to method and system for automatically generating template-based patient notes for patients admitted in a healthcare facility.

### BACKGROUND

Note taking is essential in healthcare as it helps healthcare providers (for example, doctors, clinicians, or any other healthcare professional) maintain record, facilitate transition of care, and track progress of each patient. The process typically involves manual effort in writing or typing notes by the healthcare providers or their assistants. The notes include observations and insights of a healthcare provider corresponding to a patient. In a hospital setting, the healthcare provider typically schedules a visit to multiple patients at a time (i.e., physician's round). The note taking practice helps to keep patient records more organized and for future reference to understand lab test results, diagnosis given, and the treatment prescribed to the patient.

Traditionally, the notes may be taken physically on a notepad by pen or digitally on a smartphone, a tablet, or any other electronic device that facilitates note taking. Generally, the notes are taken in a particular format. A healthcare organization (such as, a hospital) may have a standardized template for taking notes. In other cases, the healthcare providers may follow their own customized and personalized template for taking notes. However, creating templated notes from the patient data and the healthcare provider's insights (as a dictated, typed, or copied paragraph) is time consuming. Manual notes taking is also not very standardized and can vary for different healthcare providers. This can lead to misinterpretation of medical notes, slows down the healthcare provider workflow, and is inefficient.

Therefore, there is a need in the present scenario for methods for automated generation of relevant and high quality notes using standard templates.

### SUMMARY

In one embodiment, a method for automatically generating template-based patient notes is disclosed. In one example, the method includes receiving patient data from Electronic Medical Records (EMR) of the patient and an audio input corresponding to a patient from a healthcare provider. The method further includes generating primary insights text data based on the audio input through a speech-to-text conversion model or an autoregressive transformer model. The primary insights text data include a plurality of unique natural language sentences. The method further includes assigning a primary category to each of the plurality of unique natural language sentences in the primary insights text data through a set of Machine Learning (ML) models. The set of ML models is based on a Natural Language Processing (NLP) technique. The method further includes assigning at least one secondary category associated with the primary category to each of the plurality of unique natural language sentences in the primary insights text data through the set of ML models. The method further includes generating a final note corresponding to the patient including a combination of the assigned primary category and at least one secondary category, the primary insights text data, and the patient data, based on a note template.

In one embodiment, a system for automatically generating template-based patient notes is disclosed. In one example, the system includes a processor and a computer-readable medium communicatively coupled to the processor. The computer-readable medium stores processor-executable instructions, which, on execution, cause the processor to receive patient data from EMR of the patient and an audio input corresponding to a patient from a healthcare provider. The processor-executable instructions, on execution, further cause the processor to generate primary insights text data based on the audio input through a speech-to-text conversion model or an autoregressive transformer model. The primary insights text data include a plurality of unique natural language sentences. The processor-executable instructions further cause the processor to assign a primary category to each of the plurality of unique natural language sentences in the primary insights text data through a set of ML models. The set of ML models is based on an NLP technique. The processor-executable instructions, on execution, further cause the processor to assign at least one secondary category associated with the primary category to each of the plurality of unique natural language sentences in the primary insights text data through the set of ML models. The processor-executable instructions, on execution, further cause the processor to generate a final note corresponding to the patient including a combination of the assigned primary category, the at least one secondary category, the primary insights text data, and the patient data, based on a note template.

In one embodiment, a non-transitory computer-readable medium storing computer-executable instructions for automatically generating template-based patient notes is disclosed. In one example, the stored instructions, when executed by a processor, cause the processor to perform operations including receiving patient data from EMR of the patient and an audio input corresponding to a patient from a healthcare provider. The operations further include generating primary insights text data based on the audio input through a speech-to-text conversion model or an autoregressive transformer model. The primary insights text data include a plurality of unique natural language sentences. The operations further include assigning a primary category to each of the plurality of unique natural language sentences in the primary insights text data through a set of ML models. The set of ML models is based on an NLP technique. The operations further include assigning at least one secondary category associated with the primary category to each of the plurality of unique natural language sentences in the primary insights text data through the set of ML models. The operations further include generating a final note corresponding to the patient including a combination of the assigned primary category, the at least one secondary category, the primary insights text data, and the patient data, based on a note template.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system automatically generating template-based patient notes, in accordance with some embodiments.

FIG. 2 illustrates a functional block diagram of an exemplary system for automatically generating template-based patient notes, in accordance with some embodiments.

FIG. 3 illustrates a flow diagram of an exemplary process for automatically generating template-based patient notes, in accordance with some embodiments.

FIG. 4 illustrates a flow diagram of a detailed exemplary control logic for automatically generating template-based patient notes, in accordance with some embodiments.

FIG. 5 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

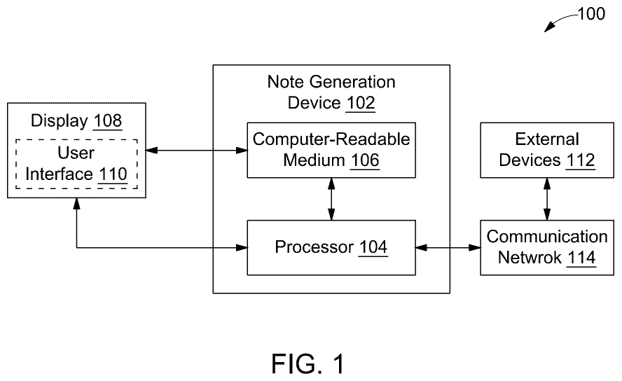

Referring now to FIG. 1, an exemplary system 100 automatically generating template-based patient notes is illustrated, in accordance with some embodiments. The system 100 may implement a note generation device 102 (for example, server, desktop, laptop, notebook, netbook, tablet, smartphone, mobile phone, smart watch, or any other computing device), in accordance with some embodiments of the present disclosure. The note generation device 102 may automatically generate template-based patient notes through a Natural Language Processing (NLP) model.

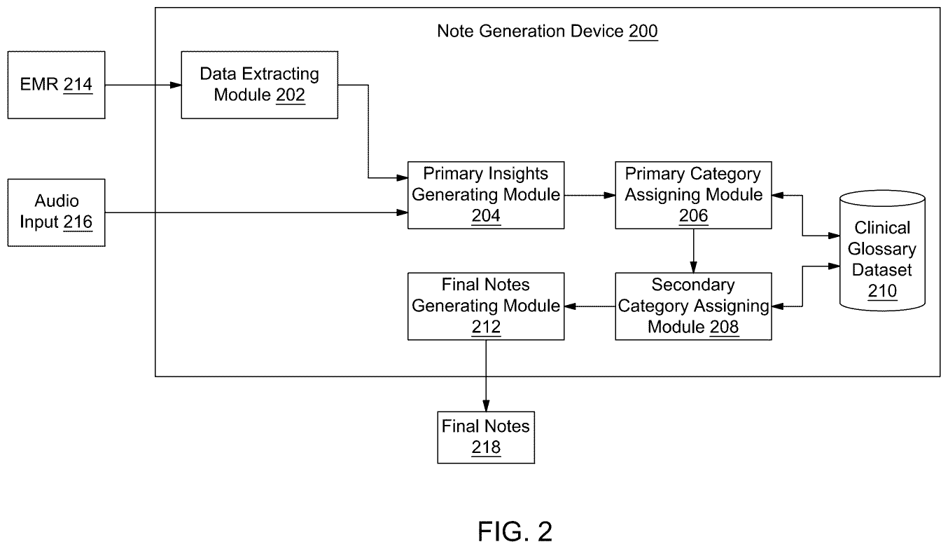

As will be described in greater detail in conjunction with FIGS. 2-4, the note generation device 102 receives patient data from Electronic Medical Records (EMR) of the patient and an audio input corresponding to a patient from a healthcare provider. The note generation device 102 further generates primary insights text data based on the audio input through a speech-to-text conversion model or an autoregressive transformer model. The primary insights text data include a plurality of unique natural language sentences. The note generation device 102 further assigns a primary category to each of the plurality of unique natural language sentences in the primary insights text data through an NLP model. The note generation device 102 further assigns at least one secondary category associated with the primary category to each of the plurality of unique natural language sentences in the primary insights text data through a set of machine learning models that leverages NLP techniques. The note generation device 102 further generates a final note corresponding to the patient including a combination of the assigned primary category, the at least one secondary category, the primary insights text data, and the patient data, based on a note template.

In some embodiments, the note generation device 102 may include one or more processors 104 and a computer-readable medium 106 (for example, a memory). The computer-readable medium 106 may include the database. Further, the computer-readable storage medium 106 may store instructions that, when executed by the one or more processors 104, cause the one or more processors 104 to automatically generate template-based patient notes, in accordance with aspects of the present disclosure. The computer-readable storage medium 106 may also store various data (for example, patient vitals, EMR, audio input of healthcare provider, clinical glossary dataset, and the like) that may be captured, processed, and/or required by the system 100.

The system 100 may further include a display 108. The system 100 may interact with a user via a user interface 110 accessible via the display 108. The system 100 may also include one or more external devices 112. In some embodiments, the note generation device 102 may interact with the one or more external devices 112 over a communication network 114 for sending or receiving various data. The external devices 112 may include, but may not be limited to, a remote server, a digital device, or another computing system.

Referring now to FIG. 2, functional block diagram of an exemplary note generation device 200 for automatically generating template-based patient notes is illustrated, in accordance with some embodiments. In an embodiment, the note generation device 200 is analogous to the note generation device 102 of the system 100. The note generation device 200 includes a data extracting module 202, a primary insights generating module 204, a primary category assigning module 206, a secondary category assigning module 208, a clinical glossary dataset 210, and a final note generating module 212. The data extracting module 202 extracts patient data from Electronic Medical Records (EMR) 214 of the patient. The patient data may include patient vitals information and patient lab test information. In an embodiment, the data extracting module 202 may receive a string of text generated from the patient data using an autoregressive transformer model. Further, the data extracting module 202 sends the patient data to the primary insights generating module 204. Additionally, the primary insights generating module 204 receives an audio input 216 corresponding to a patient from a healthcare provider.

Further, the primary insights generating module 204 generates primary insights text data based on the audio input 216 through a speech-to-text conversion model or an autoregressive transformer model. The primary insights text data include a plurality of unique natural language sentences. By way of an example, the audio input 216 may be an audio recording of a dictation of notes given by the healthcare provider in natural language. In such scenarios, the primary insights text data may be a textual transcript of the audio input 216. In some embodiments, the generated textual transcript may be further pre-processed by a rule-based data processing model or an NLP model. Further, the primary insights generating module 204 sends the primary insights text data to the primary category assigning module 206.

The primary category assigning module 206 assigns a primary category (i.e., a label) to each of the plurality of unique natural language sentences in the primary insights text data through a set of ML models. It should be noted that the set of ML models is based on an NLP technique. The primary category assigning module 206 assigns a primary category from the clinical glossary dataset 210 that includes a plurality of predefined primary categories. The plurality of predefined primary categories may be provided by a healthcare organization or may be automatically generated through an Artificial Intelligence (AI) model (for example, a large language model). In case the primary categories are generated by the AI model, the primary category assigning module 206 may provide the primary insights text data to the AI model in order to train and finetune the AI model in real-time through backpropagation. Further, the primary category assigning module 206 sends the assigned primary category and the primary insights text data to the secondary category assigning module 208.

Further, the secondary category assigning module 208 assigns at least one secondary category (i.e., a sub-label) associated with the primary category to each of the plurality of unique natural language sentences in the primary insights text data through the set of ML models. The secondary category assigning module 208 assigns a primary category from the clinical glossary dataset 210 that includes a plurality of predefined secondary categories. The plurality of predefined secondary categories may be provided by a healthcare organization or may be automatically generated through the AI model (same as that used for generating the primary categories). In case the primary categories are generated by the AI model, the secondary category assigning module 208 may provide the primary insights text data to the AI model in order to train and finetune the AI model in real-time through backpropagation. Further, the secondary category assigning module 208 sends the assigned at least one secondary category and the primary insights text data to the secondary category assigning module 208.

In some embodiments, the clinical glossary dataset 210 includes the plurality of predefined primary categories and the plurality of predefined secondary categories associated with primary categories. The assigned primary category is one of the plurality of predefined primary categories and the at least one assigned secondary category is one of the plurality of predefined secondary categories associated with the plurality of primary categories.

For each of the plurality of primary categories, there may be a plurality of associated secondary categories. Further, the clinical glossary dataset 210 and a training dataset may be used to train the set of ML models for automatic generation of template-based notes. Further, the secondary category assigning module 208 sends the assigned primary categories and the at least one associated secondary category to the final notes generating module 212.

Further, the notes generating module 212 may select a note template from a plurality of predefined note templates. Further, the notes generating module 212 generates final notes 218 corresponding to the patient including a combination of the assigned primary category, the at least one secondary category, the primary insights text data, and the patient data, based on the note template. The final notes 218 may include various sections and sub-sections as defined by the selected note template.

It should be noted that all such aforementioned modules 202-212 may be represented as a single module or a combination of different modules. Further, as will be appreciated by those skilled in the art, each of the modules 202-212 may reside, in whole or in parts, on one device or multiple devices in communication with each other. In some embodiments, each of the modules 202-212 may be implemented as dedicated hardware circuit comprising custom application-specific integrated circuit (ASIC) or gate arrays, off-the-shelf semiconductors such as logic chips, transistors, or other discrete components. Each of the modules 202-212 may also be implemented in a programmable hardware device such as a field programmable gate array (FPGA), programmable array logic, programmable logic device, and so forth. Alternatively, each of the modules 202-212 may be implemented in software for execution by various types of processors (e.g., processor 104). An identified module of executable code may, for instance, include one or more physical or logical blocks of computer instructions, which may, for instance, be organized as an object, procedure, function, or other construct. Nevertheless, the executables of an identified module or component need not be physically located together, but may include disparate instructions stored in different locations which, when joined logically together, include the module and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for automatically generating template-based patient notes. For example, the exemplary system 100 and the associated note generation device 102 may automatically generate template-based patient notes by the processes discussed herein. In particular, as will be appreciated by those of ordinary skill in the art, control logic and/or automated routines for performing the techniques and steps described herein may be implemented by the system 100 and the note generation device 102 either by hardware, software, or combinations of hardware and software. For example, suitable code may be accessed and executed by the one or more processors on the system 100 to perform some or all of the techniques described herein. Similarly, application specific integrated circuits (ASICs) configured to perform some or all of the processes described herein may be included in the one or more processors on the system 100.

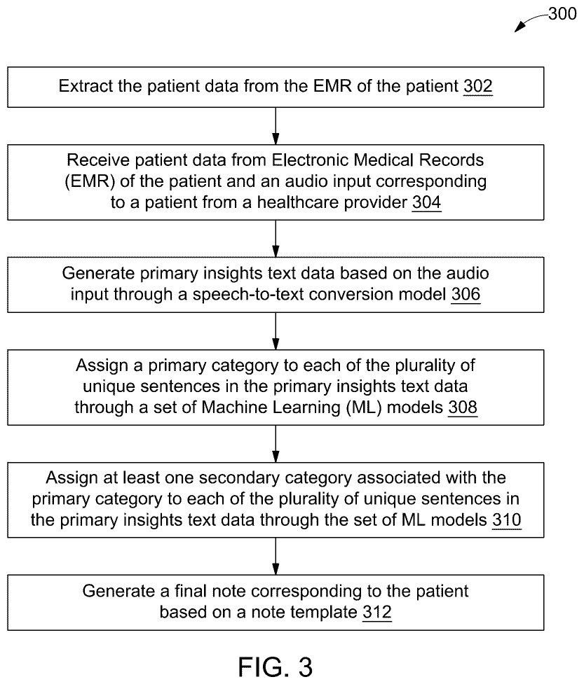

Referring now to FIG. 3, an exemplary process 300 for automatically generating template-based patient notes is depicted via a flowchart, in accordance with some embodiments. In an embodiment, the process 300 may be implemented by the note generation device 102 of the system 100. The process 300 includes extracting, by the data extracting module 202, patient data from EMR 214 of a patient, at step 302. By way of an example, the patient data may include patient vitals information and lab test information.

Further, the process 300 includes receiving, by the data extracting module 202, the patient data from the EMR of the patient and an audio input (such as, the audio input 216) corresponding to the patient from a healthcare provider, at step 304. In an embodiment, the data extracting module 202 may receive a string of text generated from the patient data using an autoregressive transformer model. By way of an example, the audio input is a dictation of notes corresponding to the patient by the healthcare provider. Further, the process 300 includes generating, by the primary insights generating module 204, primary insights text data based on the audio input 216 through a speech-to-text conversion model or an autoregressive transformer model. It should be noted that the primary insights text data include a plurality of unique natural language sentences.

Further, the process 300 includes assigning, by the primary category assigning module 206, a primary category to each of the plurality of unique natural language sentences in the primary insights text data through a set of ML models, at step 308. It should be noted that the set of ML models is based on an NLP technique. It is worth noting that the set of ML models may include one or more ML models that may not make use of NLP techniques (for example, a large language model, a deep learning model, a classification model, conversational AI model, etc.). However, there may be at least one ML model among the set of ML models that is, at least partially, based on an NLP technique. The set of ML models leverages NLP techniques to determine the primary category for each of the plurality of unique natural language sentences.

Further, the process 300 includes assigning, by the secondary category assigning module 208, at least one secondary category associated with the primary category to each of the plurality of unique natural language sentences in the primary insights text data through the set of ML models, at step 310. Additionally, the process 300 includes selecting a note template from a plurality of note templates. The plurality of note templates may be stored in a database. In an embodiment, the note template may be selected by the healthcare provider. Alternately, the note template may be a predefined standard note template for a healthcare organization. In such embodiments, each of the healthcare providers associated with the healthcare organization is provided with the predefined standard note template by default.

Further, the process 300 includes creating a clinical glossary dataset (such as, the clinical glossary dataset 210) including a plurality of predefined primary categories and a plurality of predefined secondary categories. The primary category is one of the plurality of predefined primary categories and the secondary category is one of the plurality of predefined secondary categories. In a preferred embodiment, the clinical glossary dataset is created by the healthcare organization using existing database creation tools. In another embodiment, the plurality of predefined primary categories and the plurality of predefined secondary categories is automatically generated through a pre-trained Artificial Intelligence (AI) model (for example, a large language model).

The process 300 includes generating, by the final notes generating module 212, a final note (i.e., a natural language note) corresponding to the patient including a combination of the assigned primary category, the at least one secondary category, the primary insights text data, and the patient data, based on the note template, at step 312.

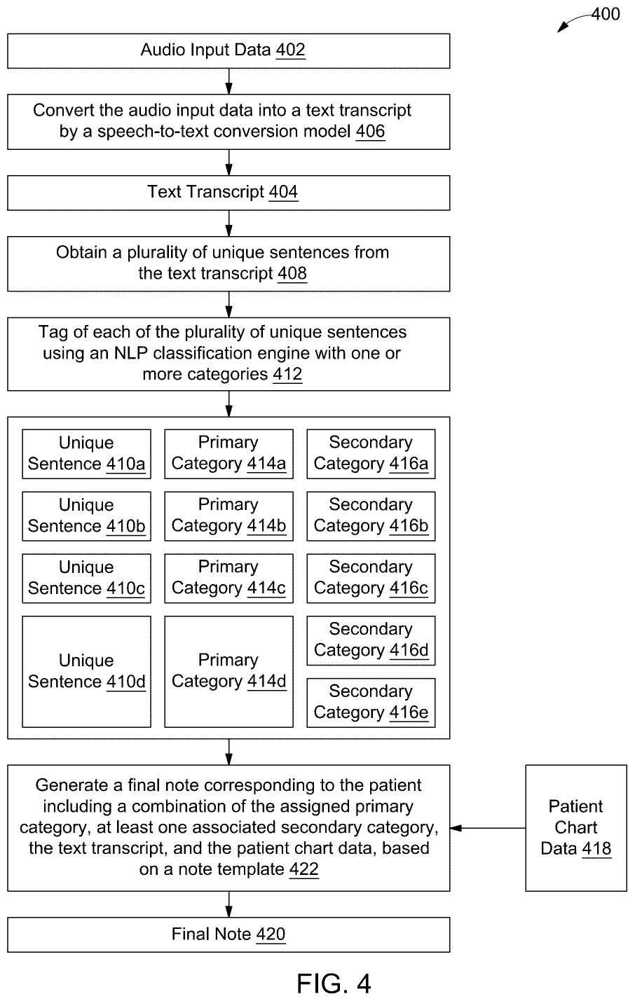

Referring now to FIG. 4, an exemplary control logic 400 for automatically generating template-based patient notes, in accordance with some embodiments. In an embodiment, the control logic 400 may be implemented by the note generation device 102 of the system 100. The note generation device 102 may receive audio input data from a healthcare provider corresponding to a patient. The control logic 400 includes converting the audio input data 402 into a text transcript 404 by a speech-to-text conversion model or an autoregressive transformer model, at step 406. Further, the control logic 400 includes obtaining a plurality of unique natural language sentences from the text transcript 404, at step 408. The plurality of unique natural language sentences may include a unique sentence 410a, a unique sentence 410b, a unique sentence 410c, and a unique sentence 410c.

By way of an example, the text transcript may be:
“24 year old male admitted with fever, altered sensorium and two episode of seizures lasting 3 minutes each. CT head normal, MRI shows meningeal enhancement. Considering his recent chemotherapy for AML, likely fungal meningitis. His absolute neutrophil count is only 109. Would start him on sepsis protocol with broadspectrum antibiotics-Cefepime 2 g every 12 hours and vancomycin 1.5 g once. Considering his elevated creatinine, would make the standing dose of Vancomycin 750 mg BID. IVF bolus 2 L per sepsis protocol stat. Would repeat BMP tomorrow morning. He is tachycardic. EKG is normal, can monitor in tele.”

For such text transcript, the plurality of unique natural language sentences is provided in table 1 below.

TABLE 1
Unique natural language sentences obtained
from an exemplary text transcript.

| Sentence No. | Sentence Value |
|---|---|
| Unique | “24 year old male admitted with fever, altered sensorium |
| sentence 1 | and two episode of seizures lasting 3 minutes each.” |
| Unique | “CT head normal, MRI shows meningeal enhancement.” |
| sentence 2 | |
| Unique | “Considering his recent chemotherapy for AML, |
| sentence 3 | likely fungal meningitis.” |
| Unique | “His absolute neutrophil count is only 109.” |
| sentence 4 | |
| Unique | “Would start him on sepsis protocol with broad spectrum |
| sentence 5 | antibiotics - Cefepime 2 g every 12 hours and vancomycin |
| | 1.5 g once.” |
| Unique | “Considering his elevated creatinine, would make the |
| sentence 6 | standing dose of Vancomycin 750 mg BID.” |
| Unique | “IVF bolus 2 L per sepsis protocol stat.” |
| sentence 7 | |
| Unique | “Would repeat BMP tomorrow morning.” |
| sentence 8 | |
| Unique | “He is tachycardic. EKG is normal, can monitor in tele.” |
| sentence 9 | |

Further, the control logic 400 includes tagging of each of the plurality of unique natural language sentences using an NLP classification engine with one or more categories, at step 412. The one or more categories include a plurality of primary categories and a plurality of secondary categories. In some embodiments, a plurality of secondary categories can be assigned to a unique natural language sentence along with a primary category. For example, the unique sentence 410a is assigned a primary category 414a and a secondary category 416a. The unique sentence 410b is assigned a primary category 414b and a secondary category 416b. The unique sentence 410c is assigned a primary category 414c and a secondary category 416c. The unique sentence 410d is assigned a primary category 414d and a secondary category 416d and a secondary category 416e.

In continuation of the example above, table 2 shows the primary categories and the secondary categories assigned to each of the plurality of unique natural language sentences in the example above.

TABLE 2
Primary categories and secondary categories assigned
to the plurality of unique natural language sentences
in the exemplary text transcript.

| Sentence No. | Primary Categories | Secondary Categories |
|---|---|---|
| Unique sentence 1 | Summary | Summary |
| Unique sentence 2 | CNS/Psych | AnP |
| Unique sentence 3 | CNS/Psych | AnP |
| | ID | |
| Unique sentence 4 | Hemat | AnP |
| Unique sentence 5 | ID | AnP, Todo |
| Unique sentence 6 | Renal | AnP, Todo |
| | ID | AnP, Todo |
| Unique sentence 7 | ID | AnP, Todo |
| Unique sentence 8 | Renal | AnP, Todo |
| Unique sentence 9 | CVS | AnP |

Additionally, the notes generation device 102 may receive patient chart data 418 from the EMR of the patient. The patient chart data 418 may include patient vitals information and patient lab test information. Further, the control logic 400 includes generating a final note 420 corresponding to the patient including a combination of the assigned primary category, at least one associated secondary category, the text transcript 404, and the patient chart data 418, based on a note template, at step 422.

In continuation of the example above, the patient chart data is represented by table 3 below.

TABLE 3
Exemplary patient chart data

| Information Type | Parameter | Value |
|---|---|---|
| Vitals | Heart Rate (HR) | 120 |
| | Respiratory Rate (RR) | 12 |
| Lab results | Sodium (Na) | 135 |
| | Potassium (K) | 3.6 |

The final note for the exemplary text transcript and the patient chart data above is as follows:
Summary: 24 year old male admitted with fever, altered sensorium and two episode of seizures lasting 3 minutes each.

### Assessment and Plan:

CNS/Psych: E4VSM6.CT head normal, MRI shows meningeal enhancement. Considering his recent chemotherapy for AML, likely fungal meningitis.
CVS: HR: 120/min BP: 110/80 mmHg. He is tachycardic. EKG is normal, can monitor in tele.
Resp: RR: 26/min, SpO2 89%, FI02 70% High Flow from Feb. 6, 2021 08:00 AM.
Abd/nutrition: Patient is on Oral-soft feeds.
Renal: Considering his elevated creatinine, would make the standing dose of Vancomycin 750 mg BID. IVF bolus 2 L per sepsis protocol stat. Would repeat BMP tomorrow morning.
ID: Cefotaxime 1 g IV every 8 hours D5. Would start him on sepsis protocol with broadspectrum antibiotics-Cefepime 2 g every 12 hours and vancomycin 1.5 g once. VF bolus 2 L per sepsis protocol stat.
Hematologic: His absolute neutrophil count is only 109.
Skin/Lymph:
Endo: Dexamethasone 8 mg IV every 8 hours D2.
Rheum/Immuno:
MSK:
Prophylaxis: Heparin 3500 IU SC every 12 hours D2.
Lines/Tubes: High Flow from Feb. 6, 2021 08:00 AM PIV, Jan. 6, 2021 09:54 PM.

### Todo:

Would start him on sepsis protocol with brospectrum antibiotics-Cefepime 2 g every 12 hours and vancomycin 1.5 g once.
Considering his elevated creatinine, would make the standing dose of Vancomycin 750 mg BID. IVF bolus 2 L per sepsis protocol stat
VF bolus 21 per sepsis protocol stat.
Would repeat BMP tomorrow morning.

As will be also appreciated, the above described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

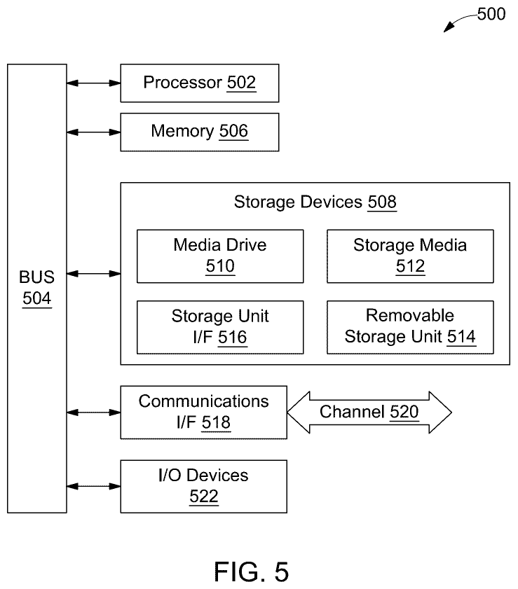

The disclosed methods and systems may be implemented on a conventional or a general-purpose computer system, such as a personal computer (PC) or server computer. Referring now to FIG. 5, an exemplary computing system 500 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 500 may represent, for example, a user device such as a desktop, a laptop, a mobile phone, personal entertainment device, DVR, and so on, or any other type of special or general-purpose computing device as may be desirable or appropriate for a given application or environment. The computing system 500 may include one or more processors, such as a processor 502 that may be implemented using a general or special purpose processing engine such as, for example, a microprocessor, microcontroller or other control logic. In this example, the processor 502 is connected to a bus 504 or other communication medium. In some embodiments, the processor 502 may be an Artificial Intelligence (AI) processor, which may be implemented as a Tensor Processing Unit (TPU), or a graphical processor unit, or a custom programmable solution Field-Programmable Gate Array (FPGA).

The computing system 500 may also include a memory 506 (main memory), for example, Random Access Memory (RAM) or other dynamic memory, for storing information and instructions to be executed by the processor 502. The memory 506 also may be used for storing temporary variables or other intermediate information during execution of instructions to be executed by the processor 502. The computing system 500 may likewise include a read only memory (“ROM”) or other static storage device coupled to bus 504 for storing static information and instructions for the processor 502.

The computing system 500 may also include a storage device 508, which may include, for example, a media drive 510 and a removable storage interface. The media drive 510 may include a drive or other mechanism to support fixed or removable storage media, such as a hard disk drive, a floppy disk drive, a magnetic tape drive, an SD card port, a USB port, a micro USB, an optical disk drive, a CD or DVD drive (R or RW), or other removable or fixed media drive. A storage media 512 may include, for example, a hard disk, magnetic tape, flash drive, or other fixed or removable medium that is read by and written to by the media drive 510. As these examples illustrate, the storage media 512 may include a computer-readable storage medium having stored therein particular computer software or data.

In alternative embodiments, the storage devices 508 may include other similar instrumentalities for allowing computer programs or other instructions or data to be loaded into the computing system 500. Such instrumentalities may include, for example, a removable storage unit 514 and a storage unit interface 516, such as a program cartridge and cartridge interface, a removable memory (for example, a flash memory or other removable memory module) and memory slot, and other removable storage units and interfaces that allow software and data to be transferred from the removable storage unit 514 to the computing system 500.

The computing system 500 may also include a communications interface 518. The communications interface 518 may be used to allow software and data to be transferred between the computing system 500 and external devices. Examples of the communications interface 518 may include a network interface (such as an Ethernet or other NIC card), a communications port (such as for example, a USB port, a micro USB port), Near field Communication (NFC), etc. Software and data transferred via the communications interface 518 are in the form of signals which may be electronic, electromagnetic, optical, or other signals capable of being received by the communications interface 518. These signals are provided to the communications interface 518 via a channel 520. The channel 520 may carry signals and may be implemented using a wireless medium, wire or cable, fiber optics, or other communications medium. Some examples of the channel 520 may include a phone line, a cellular phone link, an RF link, a Bluetooth link, a network interface, a local or wide area network, and other communications channels.

The computing system 500 may further include Input/Output (I/O) devices 522. Examples may include, but are not limited to a display, keypad, microphone, audio speakers, vibrating motor, LED lights, etc. The I/O devices 522 may receive input from a user and also display an output of the computation performed by the processor 502. In this document, the terms “computer program product” and “computer-readable medium” may be used generally to refer to media such as, for example, the memory 506, the storage devices 508, the removable storage unit 514, or signal(s) on the channel 520. These and other forms of computer-readable media may be involved in providing one or more sequences of one or more instructions to the processor 502 for execution. Such instructions, generally referred to as “computer program code” (which may be grouped in the form of computer programs or other groupings), when executed, enable the computing system 500 to perform features or functions of embodiments of the present invention.

In an embodiment where the elements are implemented using software, the software may be stored in a computer-readable medium and loaded into the computing system 500 using, for example, the removable storage unit 514, the media drive 510 or the communications interface 518. The control logic (in this example, software instructions or computer program code), when executed by the processor 502, causes the processor 502 to perform the functions of the invention as described herein.

Thus, the disclosed method and system try to overcome the technical problem of generating efficient notes of patient condition by healthcare provider. The method and system provide means to successfully generate automatic template-based patient notes. Further, the method and system increase the efficiency of placing notes by the healthcare provider for each patient.

As will be appreciated by those skilled in the art, the techniques described in the various embodiments discussed above are not routine, or conventional, or well understood in the art. The techniques discussed above provide automatically generating template-based patient notes. The techniques first receive patient data from EMR of the patient and an audio input corresponding to a patient from a healthcare provider. The techniques then generate primary insights text data based on the audio input through a speech-to-text conversion model. The primary insights text data includes a plurality of unique natural language sentences. The techniques then assign a primary category to each of the plurality of unique natural language sentences in the primary insights text data through a set of ML models. The set of ML models is based on an NLP technique. The techniques then assign at least one secondary category associated with the primary category to each of the plurality of unique natural language sentences in the primary insights text data through the set of ML models. The techniques then generate a final note corresponding to the patient including a combination of the assigned primary category, at least one secondary category, the primary insights text data, and the patient data, based on a note template.

In light of the above mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has described method and system for automatically generating template-based patient notes. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for automatically generating template-based patient notes, the method comprising:
receiving, by a note generation device, patient data from Electronic Medical Records (EMR) of the patient and an audio input corresponding to a patient from a healthcare provider;
generating, by the note generation device, primary insights text data based on the audio input through a speech-to-text conversion model or an autoregressive transformer model, wherein the primary insights text data comprise a plurality of unique natural language sentences;
assigning, by the note generation device, a primary category to each of the plurality of unique natural language sentences in the primary insights text data through a set of Machine Learning (ML) models, wherein the set of ML models is based on a Natural Language Processing (NLP) technique;
assigning, by the note generation device, at least one secondary category associated with the primary category to each of the plurality of unique natural language sentences in the primary insights text data through the set of ML models; and
generating, by the note generation device, a final note corresponding to the patient comprising a combination of the assigned primary category, the at least one secondary category, the primary insights text data, and the patient data, based on a note template.

2. The method of claim 1, comprising extracting the patient data from the EMR of the patient, wherein the patient data comprise patient vitals information and patient lab test information.

3. The method of claim 1, comprising selecting the note template from a plurality of note templates.

4. The method of claim 1, comprising creating a clinical glossary dataset comprising a plurality of predefined primary categories and a plurality of predefined secondary categories, wherein the primary category is one of the plurality of predefined primary categories, and wherein the secondary category is one of the plurality of predefined secondary categories.

5. The method of claim 4, comprising training the set of ML models using the clinical glossary dataset and a training dataset.

6. A system for automatically generating template-based patient notes, the system comprising:
a processor; and
a memory communicatively coupled to the processor, wherein the memory stores processor instructions, which when executed by the processor, cause the processor to:
receive patient data from Electronic Medical Records (EMR) of the patient and an audio input corresponding to a patient from a healthcare provider;
generate primary insights text data based on the audio input through a speech-to-text conversion model or an autoregressive transformer model, wherein the primary insights text data comprise a plurality of unique natural language sentences;
assign a primary category to each of the plurality of unique natural language sentences in the primary insights text data through a set of Machine Learning (ML) models, wherein the set of ML models is based on a Natural Language Processing (NLP) technique;
assign at least one secondary category associated with the primary category to each of the plurality of unique natural language sentences in the primary insights text data through the set of ML models; and
generate a final note corresponding to the patient comprising a combination of the assigned primary category, the at least one secondary category, the primary insights text data, and the patient data, based on a note template.

7. The system of claim 6, wherein the processor instructions, on execution, cause the processor to extract the patient data from the EMR of the patient, wherein the patient data comprise patient vitals information and patient lab test information.

8. The system of claim 6, wherein the processor instructions, on execution, cause the processor to select the note template from a plurality of note templates.

9. The system of claim 6, wherein the processor instructions, on execution, cause the processor to create a clinical glossary dataset comprising a plurality of predefined primary categories and a plurality of predefined secondary categories, wherein the primary category is one of the plurality of predefined primary categories, and wherein the secondary category is one of the plurality of predefined secondary categories.

10. The system of claim 9, wherein the processor instructions, on execution, cause the processor to train the set of ML models using the clinical glossary dataset and a training dataset.

11. A non-transitory computer-readable medium storing computer-executable instructions for automatically generating template-based patient notes, the computer-executable instructions configured for:
receiving, by a note generation device, patient data from Electronic Medical Records (EMR) of the patient and an audio input corresponding to a patient from a healthcare provider;
generating, by the note generation device, primary insights text data based on the audio input through a speech-to-text conversion model or an autoregressive transformer model, wherein the primary insights text data comprise a plurality of unique natural language sentences;
assigning, by the note generation device, a primary category to each of the plurality of unique natural language sentences in the primary insights text data through a set of Machine Learning (ML) models, wherein the set of ML models is based on a Natural Language Processing (NLP) technique;
assigning, by the note generation device, at least one secondary category associated with the primary category to each of the plurality of unique natural language sentences in the primary insights text data through the set of ML models, that leverages Natural Language Processing techniques; and
generating, by the note generation device, a final note corresponding to the patient comprising a combination of the assigned primary category, the at least one secondary category, the primary insights text data, and the patient data, based on a note template.

12. The non-transitory computer-readable medium of claim 11, wherein the computer-executable instructions are configured for extracting the patient data from the EMR of the patient, wherein the patient data comprise patient vital information and patient lab test information.

13. The non-transitory computer-readable medium of claim 11, wherein the computer-executable instructions are configured for selecting the note template from a plurality of note templates.

14. The non-transitory computer-readable medium of claim 11, wherein the computer-executable instructions are configured for creating a clinical glossary dataset comprising a plurality of predefined primary categories and a plurality of predefined secondary categories, wherein the primary category is one of the plurality of predefined primary categories, and wherein the secondary category is one of the plurality of predefined secondary categories.

15. The non-transitory computer-readable medium of claim 14, wherein the computer-executable instructions are configured for training the set of ML models using the clinical glossary dataset and a training dataset.
